(19) United States
(12) Patent Application Publication
(10) Pub. No.: US 2016/0331321 A1
(43) Pub. Date: Nov. 17, 2016

(54) ADJUSTABLE ELECTRODE OVERLAY DEVICES

(71) Applicants: Christopher O'Keefe, Columbus, OH (US); Daniel Waites, Wilmington, OH (US); Peter Bielecki, Cortland, OH (US); Terry Finklea, Upper Arlington, OH (US); James Dages, Gahanna, OH (US)

(72) Inventors: Christopher O'Keefe, Columbus, OH (US); Daniel Waites, Wilmington, OH (US); Peter Bielecki, Cortland, OH (US); Terry Finklea, Upper Arlington, OH (US); James Dages, Gahanna, OH (US)

(21) Appl. No.: 14/712,378

(22) Filed: May 14, 2015

(51) Int. Cl. A61B 5/00; A61B 5/0416

(52) U.S. Cl. CPC A61B 5/6805; A61B 5/0416; A61B 2562/227

(57) ABSTRACT

Electrode placement devices are provided. An electrode placement device includes a harness configured to be positioned on a patient and an elongated slot disposed within the harness. The elongated slot includes one or more electrode receiving portions configured to provide an electrode coupling location.

### TECHNICAL FIELD

The present specification generally relates to electrode overlay devices and, more particularly, to adjustable precordial electrode overlay devices for use with electrocardiogram (ECG) devices.

### BACKGROUND

Electrocardiogram (ECG) devices are used to monitor the electrical activity of a patient's heart. ECG devices include a plurality of electrodes that may be positioned on a patient to monitor this electrical activity. The electrodes may be positioned on the patient using an electrode placement device. However, electrode placement devices may not provide adjustability to allow desirable placement of electrodes on the patient.

Accordingly, electrode placement devices that incorporate adjustable electrode placement features may be desired to provide proportional and precise placement of precordial electrodes onto a patient.

### SUMMARY

In one embodiment, an electrode placement device includes a harness configured to be positioned on a patient and an elongated slot disposed within the harness. The elongated slot includes one or more electrode receiving portions configured to provide an electrode coupling location.

In another embodiment, an electrode placement device includes a harness configured to be positioned on a patient. An elongated slot is disposed within the harness. The elongated slot includes one or more magnetic locking portions configured to provide a magnetic electrode coupling location. A conductive band circumscribes the elongated slot. An integrated circuit having one or more lead wires is coupled to the harness. At least one lead wire is electrically coupled to the conductive band of the elongated slot.

In yet another embodiment, a precordial electrode placement device includes a harness comprising a stretchable material and a plurality of ribs disposed within the harness and extending in a patient heightwise direction. The plurality of ribs are structurally configured to facilitate proportional stretching of the harness. One or more electrode receiving portions are disposed within the harness between adjacent ribs. The one or more electrode receiving portions are configured to provide electrode coupling locations.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an example harness of an electrode placement device according to one or more embodiments shown or described herein;

FIG. 2 schematically depicts an exploded view of an example electrode for use in an electrode placement device according to one or more embodiments shown or described herein;

FIG. 3 schematically depicts another example harness of an electrode placement device according to one or more embodiments shown or described herein;

FIG. 4 schematically depicts an example harness of an electrode placement device having a plurality of ribs according to one or more embodiments shown or described herein; and

FIG. 5 schematically depicts an example vest of an electrode placement device according to one or more embodiments shown or described herein.

### DETAILED DESCRIPTION

Embodiments of the present disclosure are directed to electrode placement devices that allow for rapid and accurate placement of electrodes, for example, one or more of the electrodes of an electrocardiography (ECG) device. The electrode placement device may be precordial electrode placement devices designed to position electrodes at precordial electrode locations V1-V6 of the ECG device, for example, a 12-lead ECG device.

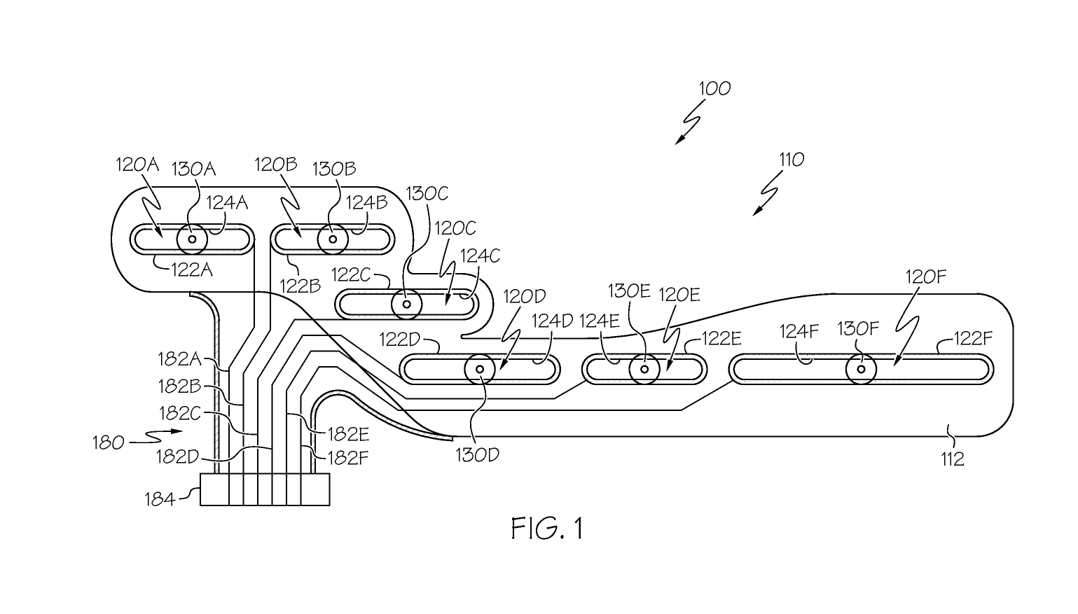

Referring now to FIG. 1, an example embodiment of the electrode placement device 100 is depicted. In this embodiment, the electrode placement device 100 comprises a harness 110 that includes one or more elongated slots 120 disposed within the harness 110. The harness 110 comprises an outward-facing surface 112 and a patient-facing surface, opposite the outward-facing surface 112. When the harness 110 is positioned on a patient, the patient-facing surface may be in contact with the patient and the outward-facing surface 112 of the harness 110 may be positioned outward the patient. The harness 110 may comprise a washable and/or stretchable material, for example, foam, mesh, polymer, composite, or the like. In some embodiments, the harness 110 may be a sterilization safe material. Further, the harness 110 may be positioned on the patient using one or more attachment regions which may include any attachment mechanism, for example, buttons, clips, latches, touch fasteners (e.g., Velcro™), or the like. Further, the electrode placement device 100 may include an attachment strap coupled to the harness 110 extendable around the patient to couple the harness 110 to the patient.

Referring still to FIG. 1, harness 110 may include six elongated slots 120A-120F disposed within the harness 110, however, it should be understood that any number of elongated slots 120 are contemplated. In some embodiments, the position of the elongated slots 120A-120F may be proximate the precordial electrode locations V1-V6, respectively, when the harness 110 is positioned on the patient. Further, the elongated slots 120A-120F may extend through the harness 110, such that an electrode 130A-130F positioned within an individual elongated slot 120A-120F extends through the harness 110 and may, in operation, contact a patient.

Referring still to FIG. 1, the elongated slots 120A-120F are elongated to allow each electrode 130A-130F positioned within the elongated slots 120A-120F to be continuously adjustable within a horizontal plane (e.g., along a length of the elongated slots 120A-120F) while remaining fixed and constrained in a vertical plane (e.g., fixed in a patient heightwise direction). In use, this continuous adjustability may account for variability in patient body size and type. For example, the continuous adjustability provided by the elongated slots 120A-120F allow each electrode 130A-130F to be positioned at each precordial electrode location V1-V6, regardless of the patient's body size. The elongated slots 120A-120F may be uniform or variable in length with respect to one another. For example, the elongated slots 120A-120F may have a length of about 5-12 cm, such as 8 cm, 10 cm, or the like. Additionally, the elongated slots 120A-120F may have a uniform height. The height of the elongated slots 120A-120F may correspond to the size of the electrodes 130A-130F such that each electrode 130A-130F may be slidably and/or removably coupled to an elongated slot 120A-120F. In some embodiments, printed scales (e.g., centimeters, or the like) are positioned on the harness 110 along the elongated slots 120A-120F. Printed scales may provide visual assistance in the placement of the one or more electrodes 130A-130F on the patient. This allows the electrodes 130A-130F to be placed at precise, desirable locations and allows the placement of the electrodes 130 to be reproduced for repeated use.

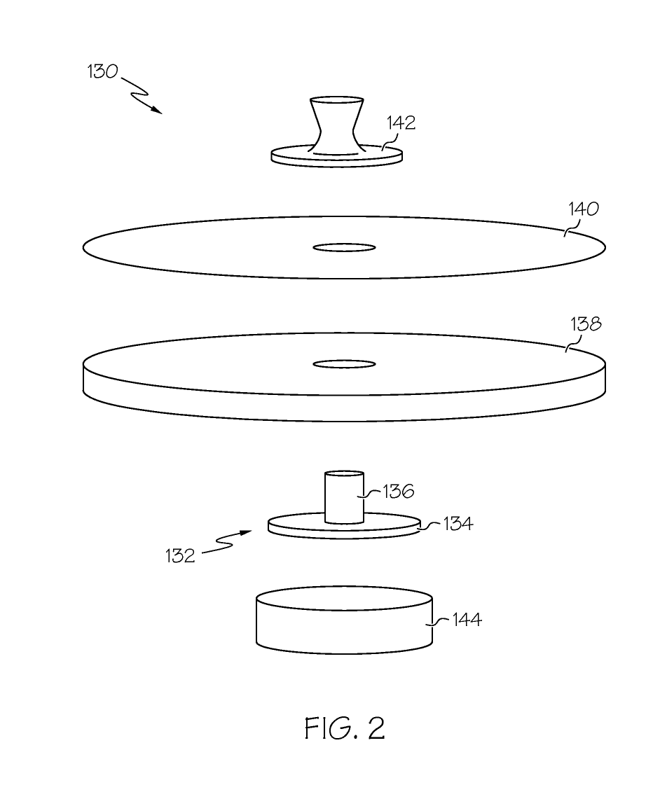

Referring now to FIG. 2, an exploded view of an example electrode 130 is depicted. While one electrode 130 is depicted, it should be understood that any number of electrodes 130 are contemplated. For example, six electrodes 130A-130F as depicted in FIG. 1, or any other number of electrodes 130. The electrode 130 may comprise an eyelet 132 having an annular portion 134 and an extending portion 136 extending from the annular portion 134. The electrode 130 may further comprises electrode foam 138 and a conducting disc 140, each comprising a hole. The conducting disc 140 may comprise a conductive material, for example, conductive metals, conductive polymers, or the like. When assembled, the extending portion 136 of the eyelet 132 may extend through the holes of both the electrode foam 138 and the conducting disc 140. Additionally, when assembled, the electrode foam 138 may be positioned between the annular portion 134 of the eyelet 132 and the conducting disc 140.

Referring still to FIG. 2, the electrode 130 further comprises a sensor stud 142 positioned on the conducting disc 140 circumscribing the hole in the conducting disc 140. In some embodiments, the sensor stud 142 may be electrically coupled to the conducting disc 140 in any configuration, for example connected to and/or secured on the conducting disc 140. Further, in some embodiments, the sensor stud 142 may extend through the hole of the conducting disc 140. When assembled, the sensor stud 142 may be electrically coupled to the extending portion 136 of the eyelet 132. Further, in some embodiments, a hydrogel 144 is positioned in contact with the annular portion 134 of the eyelet 132, for example, the surface of the annular portion 134 positioned opposite the extending portion 136. In operation, the hydrogel 144 may be placed in contact with the patient when the harness 110 is positioned on the patient. The hydrogel 144 may form an electrical signal pathway between the patient and the electrode 130. Further, the hydrogel 144 may be replaceable, for example, hydrogel 144 may be applied to the eyelet 132 before each placement of the electrode 130 in contact with the patient. Additionally, each of the components of the electrode 130 may be replaceable and/or reusable.

Referring now to FIGS. 1 and 2, each of the elongated slots 120A-120F may be circumscribed by a conductive band 122A-122F positioned along or near an edge 124A-124F of the elongated slot 120A-120F. The conductive band 122A-122F may be positioned on the patient-facing surface of the harness 110, the outward facing surface 112 of the harness 110, or both. The conductive band 122A-122F may comprise a strip of electrically conductive material, such as conductive metal, conductive polymer, or the like. As described in more detail below, the conductive band 122A-122F may be electrically coupled to one or more lead wires 182A-182F of an integrated circuit 180. Further, when the electrode 130A-130F is positioned within the elongated slot 120A-120F, the electrode 130A-130F may be electrically coupled to the conductive band 122A-122F, creating an electrical pathway between the electrodes 130A-130F and the lead wires 182A-182F. For example, the conductive band 122A-122F may maintain electrical contact (e.g., direct contact, hydrogel contact, or the like) with the sensor stud 142 and/or the conducting disc 140 of the electrode 130A-130F.

Referring still to FIGS. 1-2, individual electrodes 130A-130F may be adjustably positioned within individual elongated slots 120A-120F, for example, slidably and/or removably coupled to the elongated slots 120A-120F. The electrodes 130A-130F may be positioned within the elongated slots 120 such that a portion of the harness 110, e.g., the conductive band 122A-122F, is disposed between the electrode foam 138 and the conducting disc 140 of each electrode 130A-130F. Further, the extending portion 136 of the eyelet 132 may extend through the elongated slot 120A-120F. In some embodiments, electrode 130A-130F may be coupled to the elongated slot 120A-120F by positioning the conductive band 122 between the conducting disc 140 and the sensor stud 142. In some embodiments, the electrodes 130A-130F may be electrically coupled to the conductive band 122 in any configuration, for example, the sensor stud 142, the conducting 138, and/or the eyelet 132 may be electrically coupled to the conductive band 122 in any configuration.

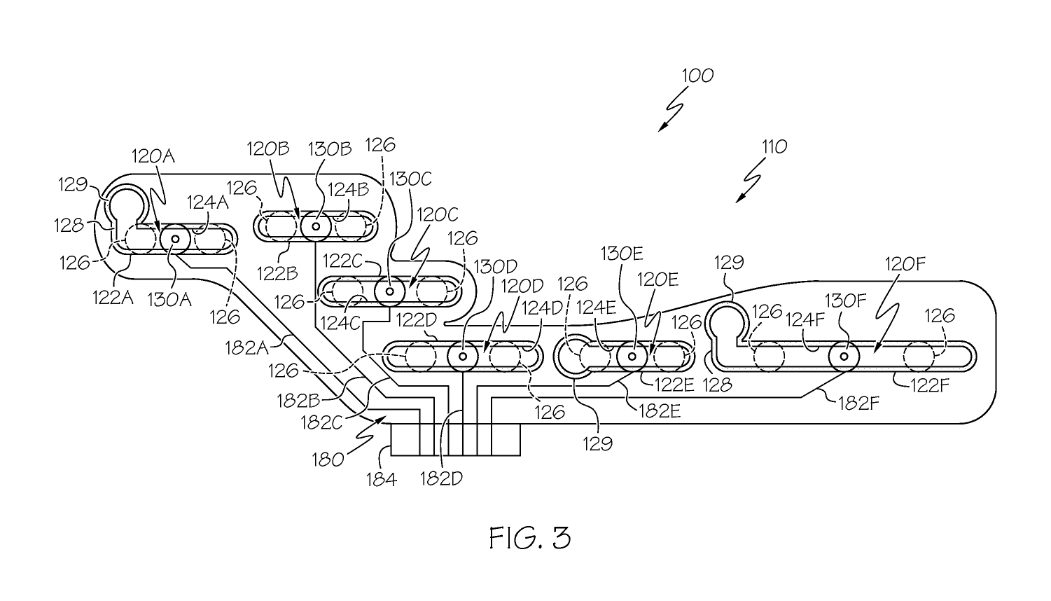

Referring now to FIG. 3, another embodiment of the electrode placement device 100 comprising one or more elongated slots 120A-120F is depicted. In some embodiments, the elongated slots 120A-120F comprise one or more electrode receiving portions configured to provide coupling locations for the one or more electrodes 130A-130F. As depicted in FIG. 3, the electrode receiving portions may comprise, for example, one or more discrete locking portions 126 and/or one or more electrode removal slots 128 having widened portions 129 which each provide coupling locations for the electrodes 130A-130F. It should be understood that each elongated slot 120A-120F may comprise one or more discrete locking portions 126 and electrode removal slots 128. Further, it should be understood that some elongated slots 120A-120F may comprise discrete locking portions 126 and not electrode removal slots 128, or vice versa. Additionally, it should be understood that the FIG. 3 is merely illustrative in depicting the various elongated slots 120A-120F having these features.

Referring now to FIGS. 2 and 3, the one or more elongated slots 120A-120F may comprise one or more discrete locking portions 126 that provide a coupling location for one or more electrodes 130A-130F within the elongated slots 120A-120F. In some embodiments, magnets and/or fasteners (e.g., snap-fit fasteners, or the like) may be embedded in the harness 110 to form each of the discrete locking portions 126 positioned along the elongated slots 120A-120F. In some embodiments, the discrete locking portions 126 include a sensor stud 142 or other electrode component(s) embedded in the harness 110, for example, within the elongated slot 120A-120F. The embedded sensor stud 142 or other electrode component(s) may be positioned in the discrete locking portion 126 with magnets and/or fasteners. In some embodiments, the embedded sensor stud 142 or other electrode component(s) may provide a snap fit location without the use of magnets and/or fasteners. In operation, an individual electrode 130 comprising each of the electrode components described above except the sensor stud 142 or other electrode component(s) may be removably coupled to the embedded sensor stud 142 or other electrode component(s) to couple the individual electrode 130 within the discrete locking portion 126.

In some embodiments, the discrete locking portions 126 may be slidably positioned within the elongated slots 120A-120F such that the discrete locking portions 126 are configured to slide along the elongated slots 120A-120F. For example, one or more discrete locking portions 126 may be positioned within one or more individual elongated slot 120A-120F and may be configured to slide along the length of the elongated slot 120A-120F. In other embodiments, multiple discrete locking portions 126 are intermittently fixed within the elongated slots 120A-120F. For example, the discrete locking portions 126 may be intermittently positioned along the elongated slot 120, for example, at uniform increments, ranging anywhere from 0.1-2 cm increments, for example, 0.5 cm, 1 cm and 1.5 cm uniform increments. For example, in one embodiment, the elongated slot 120 comprises a plurality of 0.5 cm uniform increments. In another embodiment, the elongated slot 120 comprises a plurality of 1.0 cm uniform increments. In yet another embodiment, the elongated slot 120 comprises a plurality of 1.5 cm uniform increments. These discrete locking portions 126 comprise discrete receiving locations within the elongated slots 120 such that electrodes 130 may be removably positioned within each discrete locking portion 126.

In other embodiments, the harness 110 may not include any elongated slots 120 and instead may comprise one or more discrete locking portions 126 disposed within the harness 110 at locations corresponding, for example, to the precordial electrode locations V1-V6. For example, at each precordial electrode locations V1-V6, multiple adjacent discrete locking portions 126 may be disposed within the harness 110, such that if an electrode 130 is coupled to an individual discrete locking portion 126, adjacent discrete locking portions 126 may be vacant. In embodiments without elongated slots 120, each discrete locking portion 126 may be circumscribed by a conductive band 122 that is electrically coupled to a lead wire 182. Further, in some embodiments, each discrete locking portion 126 may be directly coupled to a lead wire 182 to provide an electronic pathway between the electrode 130 positioned within the discrete locking portion 126 and a corresponding lead wire 182.

Referring still to FIG. 3, the electrode receiving portions of the elongated slots 120A-120F may also comprise one or more electrode removal slots 128. The electrode removal slots 128 may be connected to the elongated slots 120A-120F such that a pathway is formed between electrode removal slots 128 and the elongated slots 120. Further, the electrode removal slots 128 may comprise a widened portion 129 that allows electrodes 130A-130F to be removably coupled to the electrode removal slots 128. For example, an individual electrode 130 may be positioned within the widened portion 129 and placed into slidable engagement with the electrode removal slots 128 and the elongated slots 120. Further, as depicted in FIG. 3, two of the illustrated elongated slots 120 include electrode removal slots 128 that extend in a substantially patient heightwise direction along the harness 110 with respect to the elongated slots 120. In the illustrated embodiment, the widened portions 129 of the electrode removal slots 128 are positioned at the end of electrode removal slot 128, however, in alternative embodiments, the widened portions 129 may be positioned in any location along the electrode removal slots 128. Further, in other embodiments, the widened portion 129 may be positioned at an end of an individual elongated slot 120, or other location along the elongated slot 120, without an electrode removal slot 128 positioned therebetween. In this embodiment, the widened portion 129 may provide a coupling location within the elongated slot 120 for electrodes 130.

Referring now to FIGS. 1-3, additional embodiments of the electrode placement device 100 are contemplated. In some embodiments, the electrodes 130A-130F may be laminated into the harness 110. In some embodiments, eyelets 132 and sensor studs 142 may be secured onto the harness 110 using lamination or other methods. For example, the material of the harness 110 (e.g., the conductive band 122) may be positioned between the eyelet 132 and sensor stud 142. Further, tacky strips of hydrogel 144 may be laminated onto the patient-facing surface of the harness 110 in contact with conductive components of the electrode and/or conductive band and/or lead wire. Additionally, some or all of the electrode components may be removably positioned in the harness 110 to allow old or used components to be replaced with new components.

Referring still to FIGS. 1-3, the harness 110 may include an integrated circuit 180 integrated into the harness 110. The integrated circuit 180 may include one or more lead wires 182A-182F positioned within the harness 110. Each lead wire 182A-182F may be electrically coupled to a corresponding conductive band 122A-122F and may provide an electronic pathway for electronic signals measured by the electrodes 130A-130F. Further, each lead wire 182A-182F may be electrically coupled to a connection port 184 that provides a termination location for each lead wire 182A-182F. In some embodiments, the lead wires comprise ribbon wiring, supplemental wiring, or the like. In some embodiments, the lead wires 182 may be positioned within the material of the harness 110. Further, the electrode placement device 100 may include a wireless communication device to facilitate the transmission of wireless signals, such as WiFi, Bluetooth, or the like. In some embodiments, the wireless signal may be the electrical signals measured by the electrodes 130.

In some embodiments, the lead wires 182 may further include one or more circuit wire switches (e.g., on-off switches). The circuit wire switches may be positioned on a lead wire 182 that is communicatively coupled to a particular conductive band 122, discrete locking portion 126, electrode 130, or the like. In operation, the circuit wire on-off switches may selectively engage and disengage electrical signal transmission provided by a particular conductive band 122, discrete locking portion 126, and/or electrode 130. This allows the integrated circuit 180 to transmit or block signals from select conductive bands 122, discrete locking portions 126, or electrodes 130, providing additionally operational adjustability. For example, in one embodiment, an electrodes 130 may be positioned in each discrete locking portion 126 of a group of adjacent discrete locking portions 126 (for example, discrete locking portions 126 positioned at an individual precordial location V1-V6) and the lead wires 182 connected to these adjacent discrete locking portions 126 may be selectively engaged and disengaged such that only one electrode 130 of the multiple adjacent discrete locking portions 126 may provide signal at any one time. For example, the circuit wire switches may be automatically or manually engaged or disengaged to facilitate signal transmission from the electrode 130 positioned in the correct precordial location. This may be utilized for patients of varying sizes to ensure that signal is transmitted from the electrode 130 positioned in the proper location.

In some embodiments, the integrated circuit 180 may electrically couple each electrode 130 to an ECG monitoring system (e.g., a mobile ECG monitoring system) providing a electrical pathway between the electrodes 130 and the ECG monitoring system. In some embodiments, the ECG monitoring system may include a processor and one or more memory modules. The one or more memory modules may include an electrode signal algorithm. The electrode signal algorithm may be implemented by the processor of the ECG monitoring system to be in accordance with ANSI/AAMI EC-12 and achieve a combined offset instability and internal noise value with that does not exceed 150 μV under a passband of 0.15 to 100 Hz for 5 minutes. This electrode signal algorithm may be implemented for ECG tracing. Further, the electrode signal algorithm may provide signal adjustability to account for variable electrode 130 placement (i.e., variability caused by the patient's body size and type) without having to adjust the location of each electrode 130. In operation, the ECG monitoring system may be use the electrode signal to measure, for example, the rate and regularity of patient's heartbeats, the position of a patient's heart chambers, the presence of any heart damage, and the effects of drugs on the heart. Additionally, the ECG monitoring system may monitor devices used to regulate the patient's heart, for example, pacemakers.

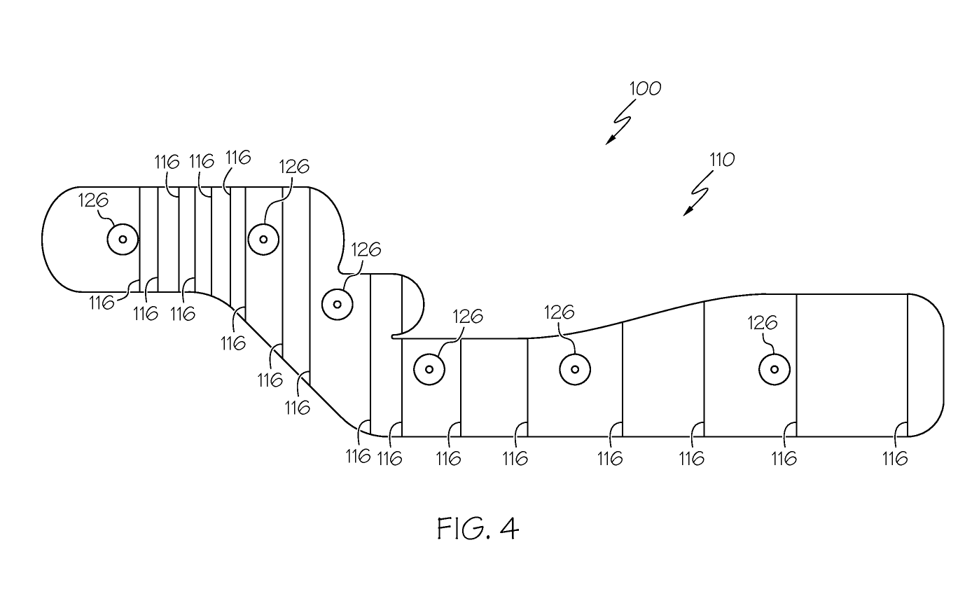

Referring now to FIG. 4, another embodiment of the electrode placement device 100 is depicted. In this embodiment, the harness 110 is made of a stretchable, material and a plurality of ribs 116 are disposed within the harness 110. In some embodiments, the harness 110 may comprise a reusable material. The plurality of ribs 116 may extend in a patient heightwise direction (e.g., in a substantially vertical direction). The plurality of ribs 116 may comprise a polymer, cured adhesive, rigid composite material, or the like. In some embodiments, the plurality of ribs 116 provide rigid barriers between discrete portions of the harness 110 positioned between adjacent ribs 116. The plurality of ribs 116 allow for controlled horizontal stretching of the harness 110 and limit vertical stretching of the harness 110. For example, individual discrete portions of the harness 110 are stretchable between adjacent ribs 116. In some embodiments, the number and thickness of ribs 116 may be used to limit the amount of stretching within discrete portions of the harness 110 while allowing more stretching within other discrete portions of the harness.

As depicted in FIG. 4, the harness 110 may comprise a plurality of electrode receiving portions, for example discrete locking portions 126 that provide coupling locations allowing one or more electrodes 130. The discrete locking portions 126 may comprise magnetic locking portions, fastener locking portions, or combinations thereof, as previously described. The discrete locking portions 126 may be disposed between adjacent ribs 116 or may extend through individual ribs 116. Further, the harness 110 having a plurality of ribs 116 may include elongated slots 120 disposed between adjacent ribs 116 or extending through individual ribs 116. It should be understood that any of the embodiments and components of the electrode placement device 100 previously described may be incorporated into the harness 110 having the plurality of ribs 116. Further, the plurality of ribs 116 may be positioned along the harness 110 to facilitate proportional stretching such that each discrete locking portion 126 may be positioned proximate the precordial electrode locations V1-V6 to fit anatomical averages of the patient. The ribs 116 may be manipulated, such as by quantity and/or thickness, to allow for more or less stretching of the harness 110 between discrete locking portions. For example, when the harness 110 is stretched around a patient, electrodes 130 positioned within discrete locking portions 126 may fall into the correct anatomical locations for precordial electrodes V1-V6 regardless of patient size.

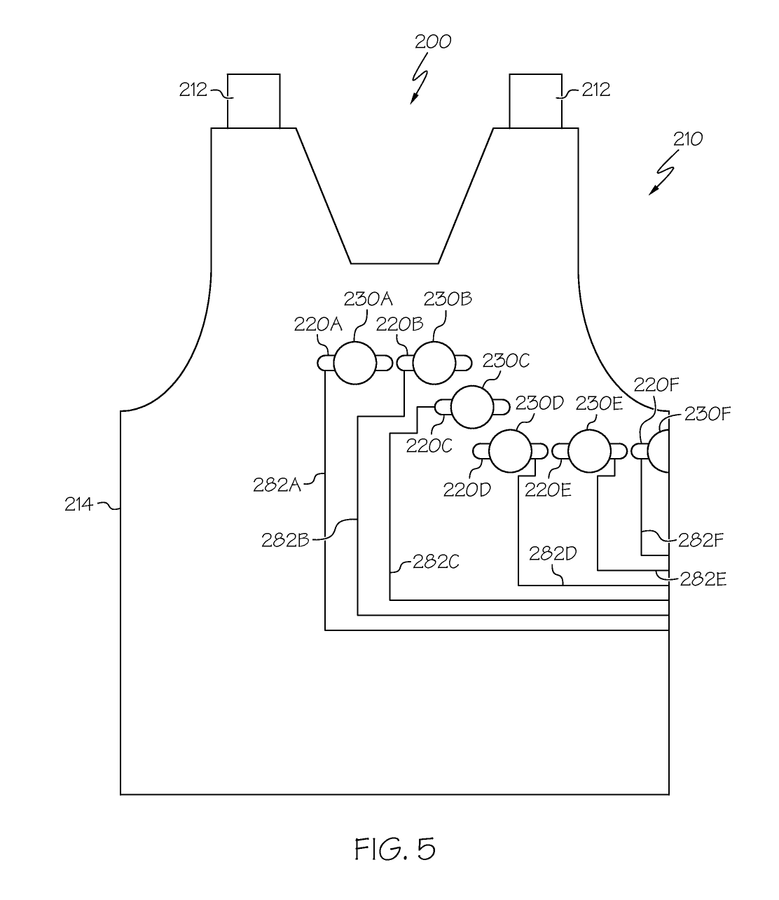

Referring now to FIG. 5, another embodiment of the electrode placement device 200 is depicted. In this embodiment, the harness of the electrode placement device 200 comprises a precordial overlay vest 210. A precordial overlay vest 210 may comprise a wearable material, for example, an elastic material, foam, mesh, polymer, composite, or the like. The precordial overlay vest 210 may include a zipper 214 for ease of application to patients, for example, patients having limited mobility. Further, the precordial overlay vest 210 may include one or more adjustable straps 212 configured to be mounted on a patient's shoulders. The adjustable shoulder straps 212 may provide the precordial overlay vest 210 with vertical adjustability.

The precordial overlay vest 210 may comprise a one or more elongated slots 220A-220F as described in the embodiments above. Further electrodes 230A-230F may be adjustably positioned within the elongated slots 220A-220F. The elongated slots 220A-220F may be positioned to correspond to electrode placement locations V1-V6, as described above. The precordial overlay vest 210 may include an integrated circuit having lead wires 282A-282F electrically coupled to the electrodes 230A-230F as described in the previous embodiments. Further, the precordial overlay vest 210 may include a pocket positioned on a patient facing side of the precordial overlay vest 210 configured to hold a device that transmits and/or electronically stores the ECG data measured by the electrodes 230A-230F. It should be understood that any of the embodiments and components of the electrode placement device 100 described above may be incorporated into the electrode placement device 200 having the precordial overlay vest 210 depicted in FIG. 5.

It should now be understood that embodiments described herein provide for electrode placement devices including stretchable and reusable harnesses and vests having elongated slots to allow electrodes to be adjustably positioned proximate a patient. The electrode placement devices include electrode receiving portions within the elongated slots to allow electrodes to be removably coupled to the elongated slots for adjustment reuse. Further, the electrode placement devices include integrated circuits to allow the electrodes to be electrically coupled to ECG monitoring devices with ease. Moreover, the electrode placement devices provide quick and precise placement of electrodes, for example precordial electrodes onto a patient.

It is noted that the term “substantially” may be utilized herein to represent the inherent degree of uncertainty that may be attributed to any quantitative comparison, value, measurement, or other representation. This term is also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. An electrode placement device comprising:
a harness configured to be positioned on a patient; and
an elongated slot disposed within the harness, wherein the elongated slot comprises one or more electrode receiving portions configured to provide an electrode coupling location.

2. The electrode placement device of claim 1 further comprising an electrode adjustably positioned within the elongated slot wherein the electrode is adjustable in one direction along a length of the elongated slot.

3. The electrode placement device of claim 1 further comprising a conductive band circumscribing the elongated slot.

4. The electrode placement device of claim 3, further comprising an electrode adjustably positioned within the elongated slot and electrically coupled to the conductive band.

5. The electrode placement device of claim 3, further comprising an integrated circuit coupled to the harness wherein the integrated circuit is electrically coupled to the conductive band of the elongated slot.

6. The electrode placement device of claim 1, wherein the harness comprises a harness material that is washable and stretchable.

7. The electrode placement device of claim 1, wherein the harness comprises a vest having one or more adjustable shoulder straps.

8. The electrode placement device of claim 1, wherein at least one of the one or more electrode receiving portions comprise a discrete locking portion.

9. The electrode placement device of claim 8, wherein the discrete locking portion is slidably coupled to the elongated slot.

10. The electrode placement device of claim 8, wherein the discrete locking portion comprises a magnetic locking portion, a fastener locking portion, or a combination thereof.

11. The electrode placement device of claim 1, wherein at least one of the one or more electrode receiving portions comprise an electrode removal slot, wherein the electrode removal slot comprises at least one widened portion.

12. The electrode placement device of claim 1 further comprising six elongated slots, wherein each individual elongated slot of the six elongated slots is disposed within the harness at a location configured to be proximate one of six precordial electrode locations V1-V6 of a patient.

13. An electrode placement device comprising:
a harness configured to be positioned on a patient;
an elongated slot disposed within the harness, wherein the elongated slot comprises one or more magnetic locking portions configured to provide a magnetic electrode coupling location;
a conductive band circumscribing the elongated slot;
an integrated circuit comprising one or more lead wires coupled to the harness wherein at least one lead wire is electrically coupled to the conductive band of the elongated slot.

14. The electrode placement device of claim 13, wherein the one or more magnetic locking portions are slidably coupled to the elongated slot.

15. The electrode placement device of claim 13 further comprising an electrode removably positioned within an individual magnetic locking portion of the elongated slot.

16. An electrode placement device comprising:
a harness comprising a stretchable material;
a plurality of ribs disposed within the harness and extending in a patient heightwise direction, wherein the plurality of ribs are structurally configured to facilitate proportional stretching of the harness; and
one or more electrode receiving portions disposed within the harness between adjacent ribs, wherein the one or more electrode receiving portions are configured to provide electrode coupling locations.

17. The electrode placement device of claim 16, wherein the one or more electrode receiving portions comprise one or more discrete locking portion that comprise magnetic locking portions, fastener locking portions, or combinations thereof.

18. The electrode placement device of claim 16, wherein the one or more electrode receiving portions further comprise one or more elongated slots.

19. The electrode placement device of claim 16 further comprising an electrode removably positioned within an individual electrode receiving portion.

20. The electrode placement device of claim 16 further comprising one or more conductive bands circumscribing the one or more electrode receiving portion.
